(19) United States
(12) Patent Application Publication
(10) Pub. No.: US 2025/0249210 A1
(43) Pub. Date: Aug. 7, 2025

(54) ACCESS SYSTEM INCLUDING GUIDE EXTENSION CATHETER AND DILATOR ADAPTED TO BE SECURED TO THE GUIDE EXTENSION CATHETER

(71) Applicant: BOSTON SCIENTIFIC SCIMED, INC., MAPLE GROVE, MN (US)

(72) Inventors: Jeffrey Steven Fuller, Brooklyn Park, MN (US); Gordon John Kocur, Lino Lakes, MN (US)

(73) Assignee: BOSTON SCIENTIFIC SCIMED, INC., MAPLE GROVE, MN (US)

(21) Appl. No.: 19/043,322

(22) Filed: Jan. 31, 2025

Related U.S. Application Data
(60) Provisional application No. 63/627,928, filed on Feb. 1, 2024.

(51) Int. Cl. A61M 25/01; A61M 25/06; A61M 29/02

(52) U.S. Cl. CPC A61M 25/0113; A61M 25/0668; A61M 29/02; A61M 2025/0681

(57) ABSTRACT

An access system adapted for accessing a desired location within a patient's vasculature includes an outer catheter such as a guide extension catheter and an inner catheter such as a dilator. The outer catheter includes an outer catheter push rod, an outer catheter sheath extending distally from the outer catheter push rod, and a first engagement feature disposed within a distal region of the outer catheter sheath. The inner catheter includes an inner catheter push rod, an inner catheter body extending distally from the inner catheter push rod, and a second engagement feature disposed within a proximal region of the inner catheter body, the second engagement feature adapted to releasably engage the first engagement feature.

### CROSS REFERENCE TO RELATED APPLICATIONS

This application claims the benefit of priority of U.S. Provisional Application No. 63/627,928 filed Feb. 1, 2024, the entire disclosure of which is hereby incorporated by reference.

### TECHNICAL FIELD

The disclosure is directed to access systems, and more particularly to access systems including a guide extension catheter and a dilator that each include features adapted to allow the dilator to remain aligned with the guide extension catheter

### BACKGROUND

Guide extension catheters may be used to enhance guide support when navigating complex, tortious anatomy. However, guide extension catheters have a distal opening that is larger than the guidewire that the guide extension catheter is being advanced along, meaning that the guide extension catheter may not exactly track the guidewire, especially when navigating a curve or a bifurcation. Physicians have developed a number of balloon-assisted tracking techniques to improve guide extension catheter tracking over a guidewire, including balloon as a rail, balloon as a dilator, inchworm and anchor balloon. Dilators have also been developed. What would be desirable is an access system including a guide extension catheter and a dilator, where the guide extension catheter and the dilator each include features that allow the dilator to remain aligned with the guide extension catheter.

### SUMMARY

This disclosure provides design, material, manufacturing method, and use alternatives for medical devices. An example may be found in an access system that is adapted for accessing a desired location within a patient's vascular. The access system includes a guide extension catheter and a dilator. The extension catheter includes a sheath extending from a distal region to a proximal region, a first push rod extending proximally from the proximal region of the sheath, and a first engagement feature disposed within the distal region of the sheath. The dilator includes a dilator body including a distal tapered region and a proximal region, a second push rod extending proximally from the tapered dilator body, and a second engagement feature disposed within the proximal region of the dilator body. The second engagement feature is adapted to releasably engage the first engagement feature.

Alternatively or additionally, the first engagement feature may include a first marker band secured about the sheath and a second marker band spaced from the first marker band and secured about the sheath. The second marker band is adapted to allow the second engagement feature to move distally past the second marker band.

Alternatively or additionally, the first marker band may have an annular shape and the second marker band may have a C-shape.

Alternatively or additionally, the second engagement feature may include an annular protrusion on the proximal region of the dilator body.

Alternatively or additionally, the second engagement feature may include an O-ring disposed on the proximal region of the dilator body.

Alternatively or additionally, the second engagement feature may include one or more bumps protruding from the proximal region of the dilator body.

Alternatively or additionally, the access system may further include a push enhancer disposed on the second push rod.

Alternatively or additionally, the push enhancer may be adapted to engage with the proximal region of the sheath.

Alternatively or additionally, the push enhancer may be positioned on the second push rod at a position that prevents the proximal region of the dilator body from moving distally beyond the distal region of the sheath.

Alternatively or additionally, the push enhancer may include a proximal collar stop having a distal region adapted to be complementary to a proximal region of the sheath.

Alternatively or additionally, the second push rod may include one or more bends that form the push enhancer.

Alternatively or additionally, the sheath may have a flared distal tip and the dilator body may be dimensioned to cover the flared distal tip.

Alternatively or additionally, the dilator body may have a hollow portion that allows the dilator body to be pulled within the sheath.

Alternatively or additionally, the access system may further include a retainer member that is adapted to be releasably secured to the first push rod and the second push rod.

Alternatively or additionally, the retainer member may include a clip.

Alternatively or additionally, the retainer member may include a peelable sheath.

Another example may be found in an access system adapted for accessing a desired location within a patient's vasculature. The access system includes a guide extension catheter and a dilator. The guide extension catheter includes a sheath extending from a distal region to a proximal region, a first push rod extending proximally from the proximal region of the sheath, a first marker band secured about the sheath, and a second marker band spaced from the first marker band and secured about the sheath. The dilator includes a dilator body including a distal tapered region and a proximal region, a second push rod extending proximally from the tapered dilator body, and a protrusion disposed within the proximal region of the dilator body that is adapted to be releasably secured between the first marker band and the second marker band.

Alternatively or additionally, the second marker band may have a C-shape that allows the second marker band to flex as the protrusion moves past the second marker band towards the first marker band.

Another example may be found in an access system adapted for accessing a desired location within a patient's vasculature. The access system includes an outer catheter and an inner catheter. The outer catheter includes an outer catheter push rod, an outer catheter sheath extending distally from the outer catheter push rod, and a first engagement feature disposed within a distal region of the outer catheter sheath. The inner catheter includes an inner catheter push rod, an inner catheter body extending distally from the inner catheter push rod, and a second engagement feature disposed within a proximal region of the inner catheter body and adapted to releasably engage the first engagement feature.

Alternatively or additionally, the outer catheter may include a guide extension catheter and the inner catheter may include a dilator adapted to extend distally from the outer catheter sheath when the inner catheter is advanced through the outer catheter.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a schematic view of an illustrative access system that includes a guide catheter and a dilator positioned proximate the guide extension catheter;

FIG. 2 is a schematic view of the illustrative access system of FIG. 1, shown with the dilator extending through the guide extension catheter;

FIG. 3 is a schematic view of an illustrative access system that includes a guide extension catheter and a dilator positioned proximate the illustrative guide extension catheter;

FIG. 4 is a schematic view of the illustrative access system of FIG. 3, shown with the dilator extending through the guide extension catheter;

FIG. 5 is a schematic view of an illustrative access system that includes a guide extension catheter and a dilator positioned proximate the illustrative guide extension catheter;

FIG. 6 is a schematic view of the illustrative access system of FIG. 5, shown with the dilator extending through the guide extension catheter;

FIG. 7 is a schematic view of an illustrative access system that includes a guide extension catheter and a dilator positioned proximate the illustrative guide extension catheter;

FIG. 8 is a schematic view of the illustrative access system of FIG. 7, shown with the dilator extending through the guide extension catheter;

FIG. 9 is a schematic view of an illustrative guide extension catheter;

FIG. 10 is a schematic view of an illustrative access system including the guide extension catheter of FIG. 9 and a dilator extending distally from the guide extension catheter;

FIG. 11 is a schematic view of the illustrative access system of FIG. 10, shown with the dilator retracted proximally into the guide extension catheter;

FIG. 12 is a schematic view of an illustrative access system, shown in combination with an illustrative retainer clip;

FIG. 13 is a schematic view of an illustrative access system, shown in combination with an illustrative retainer clip;

FIG. 14 is a schematic view of an illustrative access system, shown in combination with an illustrative peelable sheath; and

FIG. 15 is a schematic view of an illustrative access system, shown in combination with an illustrative peelable sheath.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Guide extension catheters may be used in combination with a guide catheter to extend an effective length of the guide catheter. In some instances, guide extension catheters may be used in combination with a dilator to effectively provide an atraumatic tip to the guide extension catheter. In some instances, the guide extension catheter may be considered as being an outer catheter while the dilator may be considered as being an inner catheter. Use of a dilator may also improve tracking of the guide extension catheter over a guidewire as the guide extension catheter has an inner diameter that is larger or even substantially larger than an outer diameter of the guidewire over which the guidewire extension catheter is being advanced. It will be appreciated that a guide extension catheter typically has an inner diameter that is large enough to allow other devices to be advanced through the guide catheter and the accompanying guide extension catheter. A dilator typically has a guidewire lumen extending through the dilator that is sized to accommodate the guidewire but is not substantially larger than an outer diameter of the guidewire. For example, the guidewire lumen extending through the dilator may have an inner diameter that is ten percent or less larger than an outer diameter of the guidewire.

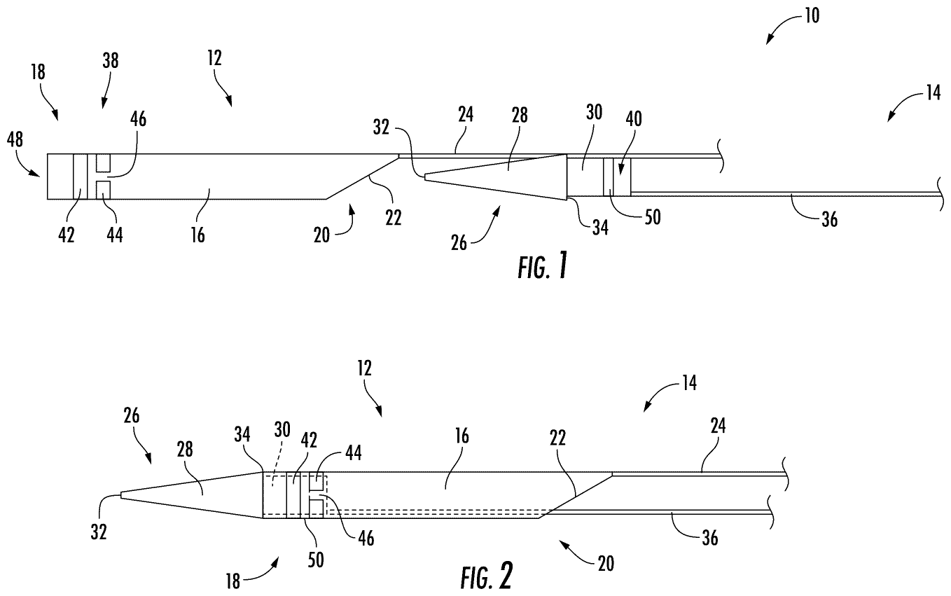

FIG. 1 is a schematic view of an illustrative access system 10 that may be used with any of a variety of different guide catheters (not shown). The illustrative access system 10 includes an outer catheter 12 and an inner catheter 14. In some instances, the outer catheter 12 may be considered as being a guide extension catheter 12. In some instances, the inner catheter 14 may be considered as being a dilator 14. In FIG. 1, the dilator 14 is positioned just proximal of the guide extension catheter 12. FIG. 2 shows the dilator 14 advanced distally into the guide extension catheter 12 in a position in which the access system 10 is ready for use.

The dilator 14 may be used in combination with the guide catheter extension 12 in navigating towards and/or through a tortuous vasculature. The guide catheter extension 12 includes a sheath 16 that may be considered as extending from a distal region 18 to a proximal region 20. As shown, the proximal region 20 may include a proximal tapered end 22, but this is not required in all cases. In some instances, the proximal region 20 may include a proximal end 22 that is not tapered. As an example, the proximal end 22 may be tapered at an angle of about 30 to 70 degrees, or about 40 to 60 degrees, or about 50 degrees plus or minus 5 degrees relative to a longitudinal extent of the sheath 16.

The guide catheter extension 12 includes a push rod 24 that extends proximally from the proximal region 20 of the sheath 16. The push rod 24 may be secured to the sheath 16 in any suitable manner. The push rod 24 may be made of metal, and may for example be welded to a metal collar (not shown) that is embedded or otherwise disposed within the sheath 16. The dilator 14 includes a dilator body 26 having a distal tapered region 28 and a proximal region 30. The distal tapered region 28 may be considered as having a conically tapering profile, although other tapering profiles are contemplated. The dilator body 26 may be a polymeric structure, and may be molded via a molding process such as injection molding. In some instances, the distal tapered region 28 has a smoothly-tapering diameter that tapers from a distal end 32 to a maximum at a point 34 that is adjacent the proximal region 30. The diameter of the distal tapered region 28 at the distal end 32, the point 34 and all portions therebetween may vary depending on the diameter of the guidewire that the dilator body 26 is intended to be advanced over as well as the diameter of the sheath 16 that the dilator body 26 will be used with. As an example, the distal end 32 may have a minimum diameter in a range of 0.017 inches to 0.020 inches. As another example, the distal end 32 may have a minimum diameter of about 0.50 millimeters (0.020 inches). The point 34 would have a diameter that ranges in accordance with the sheath 16 being part of a 5 French, 6 French or 7 French guide extension catheter. In an example, the distal tapered region 28 may have an overall length of about 15 millimeters (0.59 inches). In an example, the distal tapered region 28 may have a maximum diameter (at the point 34) that is about 1.68 millimeters (0.066 inches).

The dilator 14 includes a push rod 36. The push rod 36 may be a metal wire, for example, and may be secured to the dilator body 26 in any suitable manner. In some instances, the push rod 36 may be adhesively secured to the dilator body 26. The dilator body 26 may be over-molded onto the push rod 36. In some instances, the push rod 36 may include a shape stamped into the push rod 36 prior to over-molding the dilator body 26 over the push rod 36, with the stamped shape helping to form an interference fit between the dilator body 26 and the push rod 36. As another example, the dilator body 26 may be reflowed while being held adjacent the push rod 36. This may allow a dent formed in the outer surface of the dilator body 26 to wrap around the push rod 36. In some cases, an adhesive may also be used in addition to a reflow process. In some cases, a heat shrink tube may help to secure the dilator body 26 to the push rod 36. The push rod 36 may include a raised metal feature that increases the securement provided by over-molding. In some cases, the raised metal feature may provide a snap fit with the dilator body 26.

In some instances, the guide extension catheter 12 and the dilator 14 are adapted to be used together, and may include features that facilitate their use together. For example, the guide extension catheter 12 may include a first engagement feature 38 that is disposed within the distal region 18 of the sheath 16 and the dilator 14 may include a second engagement feature 40 that is disposed within the proximal region 30 of the dilator body 26. The first engagement feature 38 may be adapted to accept the second engagement feature 40, thereby securing the dilator 14 in position relative to the guide extension catheter 12. In some cases, the second engagement feature 40 may be considered as being adapted to releasably engage the first engagement feature 38. In some instances, the first engagement feature 38 may include a first marker band 42 and a second marker band 44 that is axially spaced from the first marker band 42. In some cases, the first marker band 42 and the second marker band 44 may be spaced about 0.5 millimeters apart in order to accommodate the second engagement feature 40 therebetween. The first marker band 42 may have an annular shape. The second marker band 44 may have an annular form but with a section 46 removed, thereby providing the second marker band 44 with a C-shaped profile.

In some instances, the first marker band 42 and the second marker band 44 may each be disposed on an outer surface of the sheath 16. The first marker band 42 and the second marker band 44 may each have an inner diameter that is less than an outer diameter of the sheath 16, meaning that the first marker band 42 and the second marker band 44 each partially compress the sheath 16. As a result, a lumen 48 extending through the sheath 16 may exhibit a first diameter decrease corresponding to where the first marker band 42 is located and a second diameter decrease corresponding to where the second marker band 44 is located. In some cases, it is the first diameter decrease and the second diameter decrease that serves to fixate the second engagement feature 40 therebetween. Because the second marker band 44 has a C-shaped profile, the second marker band 44 is able to flex when the dilator 14 is advanced distally within the lumen 48 and the second engagement feature 40 passes the second marker band 44.

In some instances, the second engagement feature 40 may include an annular protrusion 50 that is disposed within the proximal region 30 of the dilator body 26. The annular protrusion 50 may be molded into the dilator body 26. In some instances, the annular protrusion 50 may actually be a rigid O-ring that is disposed about the dilator body 26. While not shown, the proximal region 30 of the dilator body 26 may include one or more recesses that are adapted to secure the rigid O-ring in place. The annular protrusion 50 may be dimensioned to fit between the first marker band 42 and the second marker band 44, for example. As a result, the dilator 14 may be moved distally into the lumen 48 defined by the sheath 16. The first engagement feature 38 (such as the first marker band 42 and the second marker band 44) may be positioned in the distal region 18 of the sheath 16 such that as the distal tapered region 28 extends distally from the sheath 16, as seen in FIG. 2, the second engagement feature 40 snaps into position between the first marker band 42 and the second marker band 44. As a result, the guide extension catheter 12 and the dilator 14 may move together as an assembly.

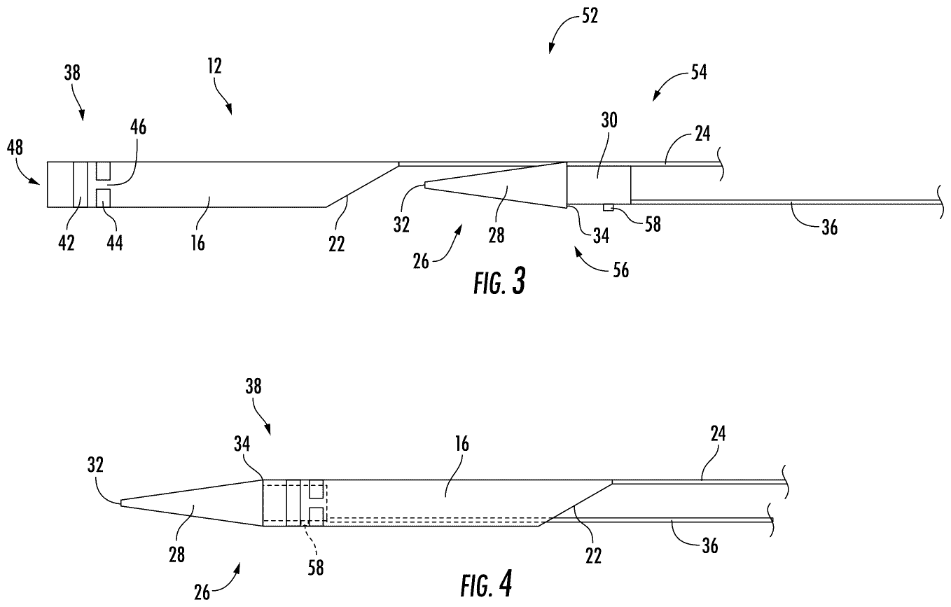

FIG. 3 is a schematic view of an illustrative access system 52 that is similar to the access system 10, but includes a slightly different dilator. The access system 52 includes the guide catheter extension 12 as discussed with respect to FIGS. 1 and 2 and a dilator 54. In FIG. 3, the dilator 54 is positioned just proximal of the guide extension catheter 12. FIG. 4 shows the dilator 54 advanced distally into the guide extension catheter 12 in a position in which the access system 52 is ready for use. The dilator 54, like the dilator 14, includes the dilator body 26 having the distal tapered region 28 and the proximal region 30. In some instances, the distal tapered region 28 has a smoothly-tapering diameter that tapers from a minimum diameter at the distal end 32 to a maximum diameter at the point 34 proximate the proximal region 30 and thus may have a conical shape. The dilator 54 includes a push rod 36. The push rod 36 may be secured to the dilator body 26 in any suitable manner. The dilator 54 includes a second engagement feature 56 that is adapted to releasably engage the first engagement feature 38. In some instances, the second engagement feature 56 may include one or more bumps 58 that are adapted to engage the first engagement feature 38. Rather than an annular protrusion (as seen in FIG. 1), the second engagement feature 56 may include one or more distinct protrusions. The second engagement feature 56 may include any number of bumps 58, although only one is shown. Each of the one or more bumps 58 may be integrally molded as part of the dilator body 26. In some instances, each of the one or more bumps 58 may be separately formed and then subsequently secured to the dilator body 26, such as adhesively.

Each of the one or more bumps 58 may be dimensioned to fit between the first marker band 42 and the second marker band 44, for example. As a result, the dilator 14 may be moved distally into the lumen 48 defined by the sheath 16. The first engagement feature 38 (such as the first marker band 42 and the second marker band 44) may be positioned in the distal region 18 of the sheath 16 such that as the distal tapered region 28 extends distally from the sheath 16, as seen in FIG. 4, the second engagement feature 56 (such as the one or more bumps 58) snaps into position between the first marker band 42 and the second marker band 44. As a result, the guide extension catheter 12 and the dilator 14 may move together as an assembly.

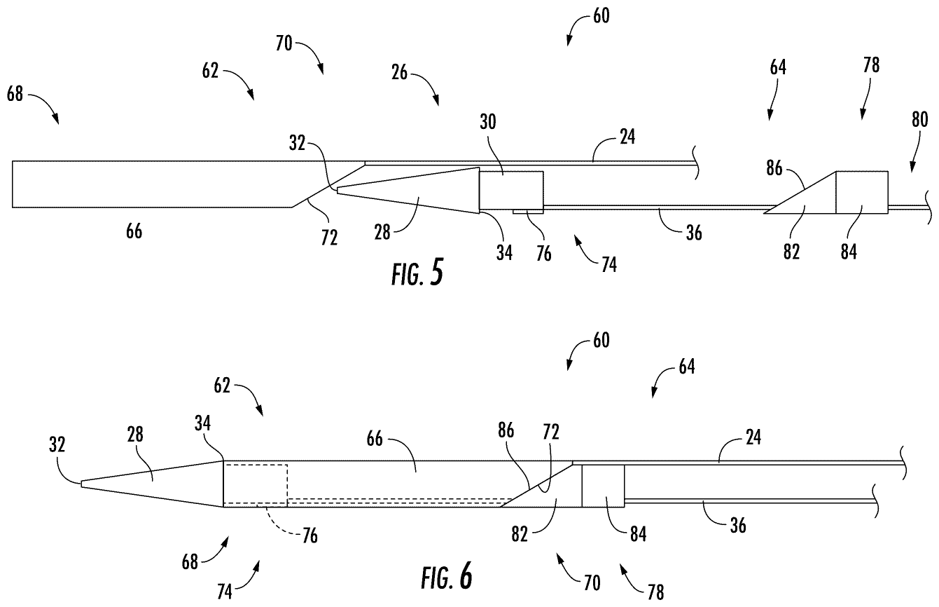

FIG. 5 is a schematic view of an illustrative access system 60 that may be used with any of a variety of different guide catheters (not shown). The illustrative access system 60 includes an outer catheter 62 and an inner catheter 64. In some instances, the outer catheter 62 may be considered as being a guide extension catheter 62. In some instances, the inner catheter 64 may be considered as being a dilator 64. In FIG. 5, the dilator 64 is positioned just proximal of the guide extension catheter 62. FIG. 6 shows the dilator 64 advanced distally into the guide extension catheter 62 in a position in which the access system 60 is ready for use.

The dilator 64 may be used in combination with the guide catheter extension 62 in navigating towards and/or through a tortuous vasculature. The guide catheter extension 62 includes a sheath 66 that may be considered as extending from a distal region 68 to a proximal region 70. In some instances, the proximal region 70 may include a proximal tapered end 72, but this is not required in all cases. The guide catheter extension 62 includes the push rod 24 extending proximally from the proximal region 70 of the sheath 66. The push rod 24 may be secured to the sheath 66 in any suitable manner. The dilator 64 includes the dilator body 26 having the distal tapered region 28 and the proximal region 30. In some instances, the distal tapered region 28 has a smoothly-tapering diameter that tapers from a minimum diameter at a distal end 32 to a maximum diameter at a point 34 proximate the proximal region 30 and thus has a conical shape. The dilator 64 includes a push rod 36. The push rod 36 may be secured to the dilator body 26 in any suitable manner.

In some instances, the guide extension catheter 62 and the dilator 64 are adapted to be used together, and may include features that facilitate their use together. For example, the guide extension catheter 62 may include a first engagement feature (not shown) that is disposed within the distal region 68 of the sheath 66 and the dilator 64 may include a second engagement feature 74 that is disposed within the proximate region 30 of the dilator body 26. In some instances, the first engagement feature may be similar to the first engagement feature 38 shown in FIGS. 1 through 4. In some instances, the second engagement feature 74 may include a protrusion 76 that is similar to the one or more bumps 58 shown in FIGS. 3 and 4. The first engagement feature may be adapted to accept the second engagement feature 74, thereby securing the dilator 64 in position relative to the guide extension catheter 62. In some cases, the second engagement feature 74 may be considered as being adapted to releasably engage the first engagement feature.

In some instances, the dilator 64 may include a push enhancer 78 that is disposed on the second push rod 36. The push enhancer 78 may help with transferring a push force from the second push rod 36 to the guide extension catheter 62. In some cases, the push enhancer 78 may also help to keep the distal tapered region 28 of the dilator body 26 aligned with the distal region 68 of the sheath 66, and prevent the distal tapered region 28 of the dilator body 26 from moving distally of the distal region 68 of the sheath 66. The push enhancer 78 may take a variety of forms, and may be secured to the second push rod 36 in any suitable manner. For example, the push enhancer 78 may be made of a polymer, and may be adhesively secured to the second push rod 36. In some cases, using an adhesive to secure the push enhancer 78 to the second push rod 36 allows for customization in the exact positioning of the push enhancer 78 relative to the second push rod 36. In some cases, the push enhancer 78 may be over-molded onto the second push rod 36. In some instances, the push enhancer 78 may be secured to the second push rod 36 at a position that corresponds to the distal tapered region 28 of the dilator body 26 being appropriately aligned with the distal region 68 of the sheath 66.

In some cases, the push enhancer 78 may be a proximal collar stop 78 that includes a collar stop body 80 that is secured to the second push rod 36. The collar stop body 80 includes an angled distal portion 82 and a proximal portion 84. The angled distal portion 82 defines an angled distal surface 86. In some instances, the angled distal surface 86 of the push enhancer 78 matches the proximal tapered end 72 of the sheath 66. In some cases, the angled distal surface 86 of the push enhancer 78 may not match the angle of the proximal tapered end 72. The angled distal portion 82 and/or the proximal portion 84 of the collar stop body 80 may be larger in diameter than the proximal tapered end 72, providing that the diameters of the angled distal portion 82 and/or the proximal portion 84 of the collar stop body 80 fit within any guide catheter that the access system 60 is being used with. As seen in FIG. 6, the angled distal surface 86 of the push enhancer 78 aligns with the proximal tapered end 72 of the sheath 66. As a result, a push force being applied to the second push rod 36 will be transferred to the sheath 66 of the guide extension catheter 62.

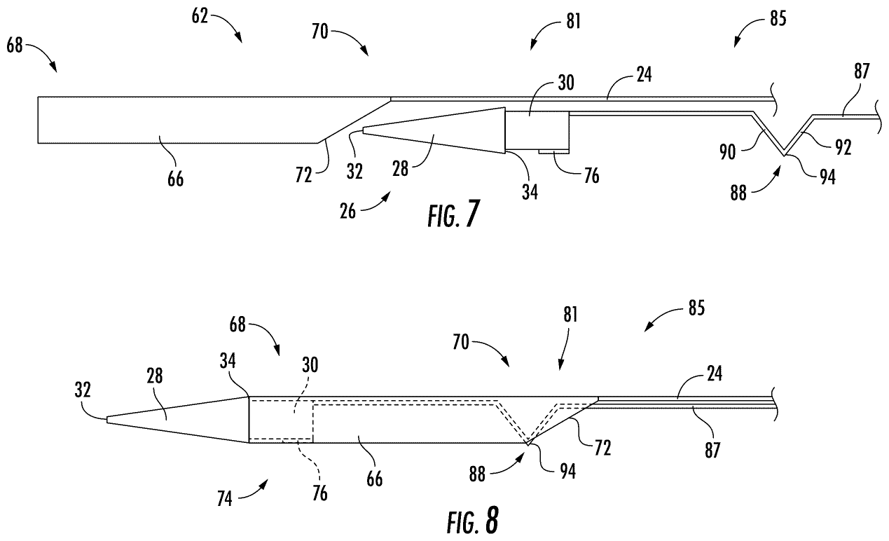

FIG. 7 is a schematic view of an illustrative access system 81 that may be used with any of a variety of different guide catheters (not shown). The illustrative access system 81 includes the outer catheter 62 as described with respect to FIGS. 5 and 6 and an inner catheter 85. In some instances, the outer catheter 62 may be considered as being a guide extension catheter 62. In some instances, the inner catheter 85 may be considered as being a dilator 85. In FIG. 7, the dilator 85 is positioned just proximal of the guide extension catheter 62. FIG. 8 shows the dilator 85 advanced distally into the guide extension catheter 62 in a position in which the access system 81 is ready for use.

The dilator 85 may be used in combination with the guide catheter extension 62 in navigating towards and/or through a tortuous vasculature. The dilator 85 includes the dilator body 26 having the distal tapered region 28 and the proximal region 30. In some instances, the distal tapered region 28 has a smoothly-tapering diameter that tapers from a minimum diameter at a distal end 32 to a maximum diameter at a point 34 proximate the proximal region 30. The dilator 85 includes a push rod 87. The push rod 87 may be secured to the dilator body 26 in any suitable manner. The push rod 87 may be a metal wire, for example, and may be secured to the dilator body 26 in any suitable manner. In some instances, the push rod 36 may be adhesively secured to the dilator body 26. The dilator body 26 may be over-molded onto the push rod 87. In some instances, the push rod 87 may include a shape stamped into the push rod 36 prior to over-molding the dilator body 26 over the push rod 87, with the stamped shape helping to form an interference fit between the dilator body 26 and the push rod 87. As another example, the dilator body 26 may be reflowed while being held adjacent the push rod 87. This may allow a dent formed in the outer surface of the dilator body 26 to wrap around the push rod 87. In some cases, an adhesive may also be used in addition to a reflow process. In some cases, a heat shrink tube may help to secure the dilator body 26 to the push rod 87. The push rod 87 may include a raised metal feature that increases the securement provided by over-molding. In some cases, the raised metal feature may provide a snap fit with the dilator body 26.

In some instances, the guide extension catheter 62 and the dilator 85 are adapted to be used together, and may include features that facilitate their use together. For example, the guide extension catheter 62 may include a first engagement feature (not shown) that is disposed within the distal region 68 of the sheath 66 and the dilator 85 may include the second engagement feature 74 that is disposed within the proximate region 30 of the dilator body 26. In some instances, the first engagement feature may be similar to the first engagement feature 38 shown in FIGS. 1 through 4. In some instances, the second engagement feature 74 may include the protrusion 76 that is similar to the one or more bumps 58 shown in FIGS. 3 and 4. The first engagement feature may be adapted to accept the second engagement feature 74, thereby securing the dilator 85 in position relative to the guide extension catheter 62. In some cases, the second engagement feature 74 may be considered as being adapted to releasably engage the first engagement feature.

In some instances, the dilator 85 may include a push enhancer 88 that is disposed on the second push rod 87. The push enhancer 88 may help with transferring a push force from the second push rod 87 to the guide extension catheter 62. In some cases, the push enhancer 88 may also help to keep the distal tapered region 28 of the dilator body 26 aligned with the distal region 68 of the sheath 66, and prevent the distal tapered region 28 of the dilator body 26 from moving distally of the distal region 68 of the sheath 66. The push enhancer 88 may take a variety of forms, and may be secured to the second push rod 87 in any suitable manner. In some instances, the push enhancer 88 may be secured to the second push rod 87 at a position that corresponds to the distal tapered region 28 of the dilator body 26 being appropriately aligned with the distal region 68 of the sheath 66.

In some instances, the push enhancer 88 may be separately formed and then secured in place to the second push rod 87. In some cases, as shown, the push enhancer 88 includes one or more bends that are formed within the second push rod 87. The push enhancer 88 may include a first segment 90 and a second segment 92, with the first segment 90 and the second segment 92 meeting at an apex 94. In some cases, it is the apex 94 that interacts with a lower (in the illustrated orientation) corner of the proximal tapered end 72 of the sheath 66. This can be seen, for example, in FIG. 8. As a result, a push force being applied to the second push rod 87 will be transferred to the sheath 66 of the guide extension catheter 62.

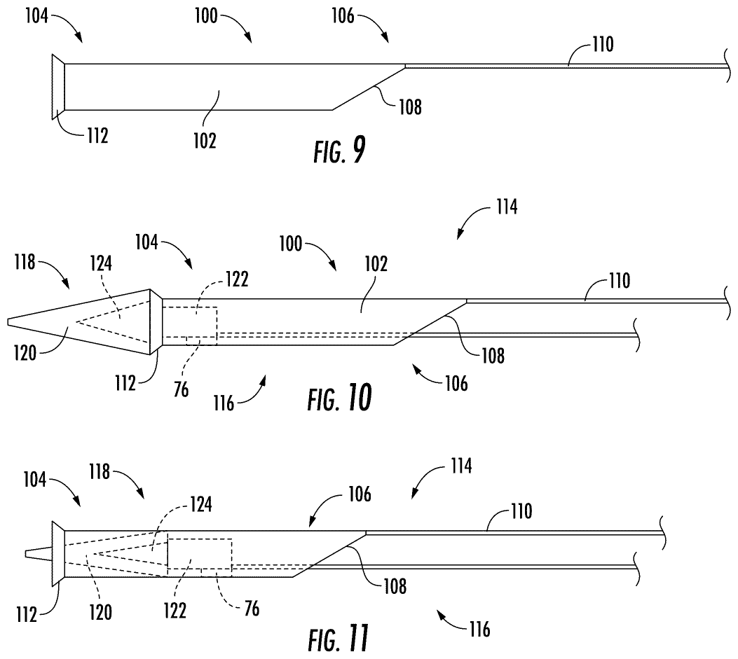

In some instances, there may be a desire to be able to withdraw a large deflated balloon into the guide extension catheter. FIG. 9 is a schematic view of an illustrative guide extension catheter 100 that includes a sheath 102 that may be considered as extending from a distal region 104 to a proximal region 106. In some instances, the proximal region 106 may include a proximal tapered end 108, but this is not required in all cases. The guide catheter extension 100 includes a push rod 110 that extends proximally from the proximal region 106 of the sheath 102. The push rod 110 may be secured to the sheath 102 in any suitable manner. The guide extension catheter 100 includes a flared distal tip 112 that is located within the distal region 104 of the sheath 102. As will be appreciated, the flared distal tip 112 may make it easier to withdraw deflated (but previously inflated) balloons into the sheath 102.

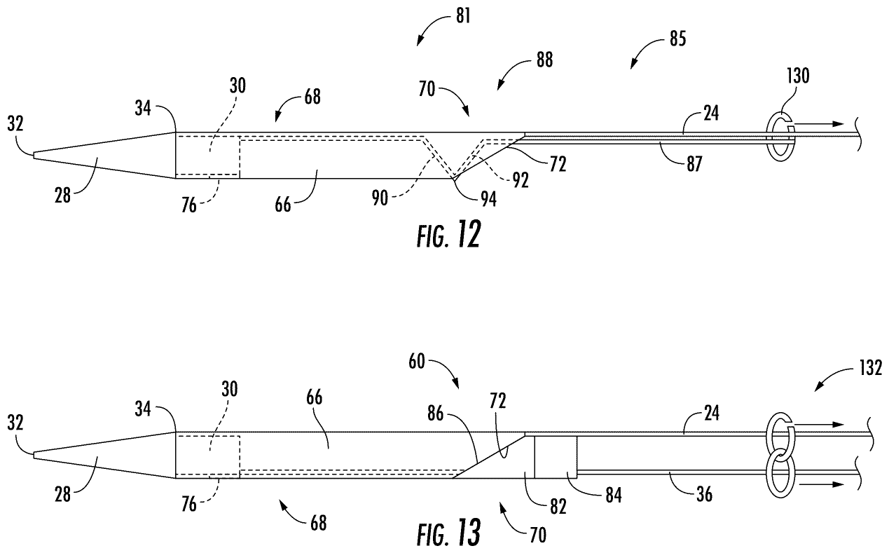

In some instances, the guide extension catheter 100 may be used in combination with a dilator that is adapted to accommodate the flared distal tip 112 of the sheath 102. FIG. 10 is a schematic view of an illustrative access system 114 that includes the guide extension catheter 100 in combination with a dilator 116. The dilator 116 includes a dilator body 118 that has a distal tapered region 120 and a proximal region 122. In some instances, the distal tapered region 120 may have a conical shape. In some cases, the dilator body 118 further includes a hollow region 124 that is in addition to a guidewire lumen (not shown) extending through the dilator body 118. In some instances, as seen in FIG. 10, the distal tapered region 120 has a maximum diameter that, while matching an outer diameter of the flared distal tip 112, is larger than a diameter of the sheath 102. In some cases, the hollow region 124 allows the distal tapered region 120 to reduce its maximum diameter as the dilator 116 is withdrawn proximally into the sheath 102, as seen in FIG. 11. In some cases, the hollow region 124 may not be hollow, but may represent a foam portion of the dilator body 118, for example.

The dilator 116 is shown as including the protrusion 76 that is similar to the one or more bumps 58. The dilator 116 may include the annular protrusion 50 instead of the protrusion 76. While not shown, the guide extension catheter 100 may include the first engagement feature 38 shown in FIGS. 1 through 4, such as the first marker band 42 and the second marker band 44. In some instances, the dilator 116 may include the push enhancer 78 shown for example in FIGS. 5 and 6, or the push enhancer 88 shown in FIGS. 7 and 8. In some instances, the guide extension catheter 12 or the guide extension catheter 62 may include the flared distal tip 112. In some cases, the dilator 14 or the dilator 64 may include the hollow region 124, or otherwise be compressible, in order to allow the dilator body 26 or the dilator body 118 to compress when withdrawn proximally into the guide extension catheter.

In some cases, there may be a desire to hold the push rod 24 (of the guide extension catheter 12, for example) and the push rod 36 (of the dilator 14, for example) together when advancing or withdrawing the access system 10, the access system 60 or the access system 81. FIG. 12 shows the access system 81 shown in combination with a push rod retainer clip 130.

The push rod retainer clip 130 may be shaped like a figure eight, or like the mathematical symbol ∞ (infinity).

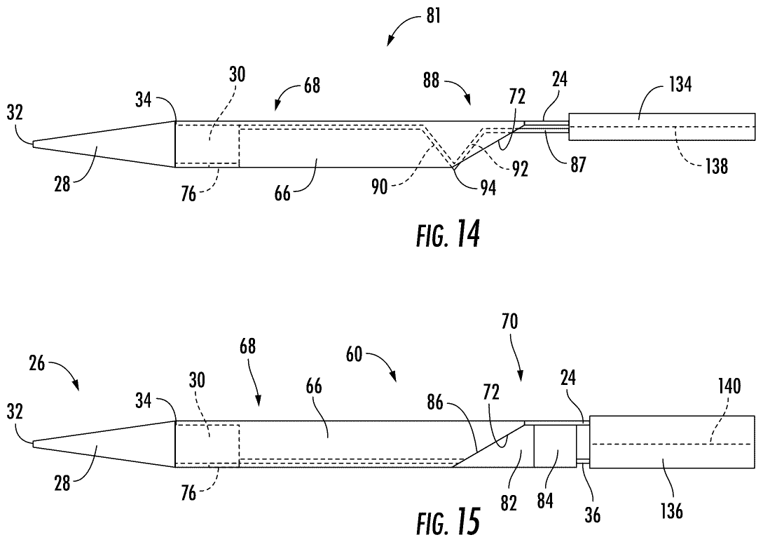

In some instances, the push rod retainer clip 130 may be adapted to allow the push rod retainer clip 130 to be removed from the push rods 24 and 87 once the access control system 81 has been disposed within a guide catheter. FIG. 13 shows the access system 60 shown in combination with the push rod retainer clip 132. In some cases, the push rod retainer clip 132 would remain outside of any hemostasis valve, and would slide relative to the push rods 24 and 36 (as shown). While not shown, the push rod retainer clip 130 and/or the push rod retainer clip 132 may also be used in combination with the access system 10.

In some cases, the push rod retainer clip 130 may be dimensioned to remain outside of any hemostasis valve, and may be adapted to slide relative to the push rods 24 and 87 as the push rods 24 and 87 are advanced through the hemostasis valve. The push rod retainer clip 130 is adapted to keep the two push rods 24 and 87 from getting too far from each other. As an example, the hemostasis valve may have a size of 10 French, or a diameter of about 3.5 millimeters (0.14 inches), and thus the push rod retainer clip 130 may have a maximum dimension that is in a range of about 5 millimeters (0.20 inches) to about 10 millimeters (0.39 inches). The push rod retainer clip 130 may be made of a variety of different materials including metal or hard plastic. In some cases, the push rod retainer clip 130 may be made of polycarbonate, polyether block amides such as those available commercially under the Pebax™ name, and ABS (acrylonitrile butadiene styrene). In some cases, the push rod retainer clip 130 may be made of a polymer such as polyethylene.

FIG. 14 shows the access system 81 in combination with a peelable sheath 134. In some cases, the peelable sheath 134 may be used to hold the push rods 24 and 87 together and prevent the push rods 24 and 87 from wrapping around each other when advancing or withdrawing the access system 10, the access system 60 or the access system 81. In some cases, the peelable sheath 134 would have an outer diameter that is greater than 0.1 inches, and thus will remain outside of any hemostasis valve, and would slide relative to the push rods 24 and 87. In some cases, the peelable sheath 134 is a polymeric tube that includes a weakened line 138 that may facilitate peeling the peelable sheath 134 away. The peelable sheath 134 may have any suitable length, ranging from perhaps 0.25 inches to several inches. As an example, the peelable sheath 134 may have a diameter that is in a range of about 5 millimeters to about 10 millimeters.

In some cases, the peelable sheath 134 may include one or more tabs that may be grasped to facilitate peeling away the peelable sheath 134. The peelable sheath 134 may be formed of any suitable polymer. In some cases, wall thickness may be adjusted to accommodate the strength properties of a particular polymer.

FIG. 15 shows the access system 60 in combination with a peelable sheath 136. In some cases, the peelable sheath 136 may be used to hold the push rods 24 and 36 together when advancing or withdrawing the access system 10, the access system 60 or the access system 81. In some cases, the peelable sheath 136 would remain outside of any hemostasis valve, and would slide relative to the push rods 24 and 36. In some cases, the peelable sheath 136 is a polymeric tube that includes a weakened line 140 that may facilitate peeling the peelable sheath 136 away. The peelable sheath 136 may have any suitable length, ranging from perhaps 0.25 inches to several inches. The peelable sheath 136 may have any suitable length, ranging from perhaps 0.25 inches to several inches. As an example, the peelable sheath 136 may have a diameter that is in a range of about 5 millimeters to about 10 millimeters. In some cases, the peelable sheath 136 may include one or more tabs that may be grasped to facilitate peeling away the peelable sheath 136. The peelable sheath 136 may be formed of any suitable polymer. In some cases, wall thickness may be adjusted to accommodate the strength properties of a particular polymer. While not shown, the peelable sheath 134 and/or the peelable sheath 136 may also be used in combination with the access system 10.

The materials that can be used for the various components of the devices disclosed herein may include those commonly associated with medical devices. The various components of the devices/systems disclosed herein may include a metal, metal alloy, polymer (some examples of which are disclosed herein), a metal-polymer composite, ceramics, combinations thereof, and the like, or other suitable material. Some examples of suitable metals and metal alloys include stainless steel, such as 304V, 304 L, and 316 LV stainless steel; mild steel; nickel- titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium- molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of the various components of the devices disclosed herein may be doped with, made of, or otherwise include a radiopaque material. Radiopaque materials are understood to be materials capable of producing a relatively bright image on a fluoroscopy screen or another imaging technique during a medical procedure. This relatively bright image aids the user of the device in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An access system adapted for accessing a desired location within a patient's vasculature, the access system comprising:
a guide extension catheter including:
a sheath extending from a distal region to a proximal region;
a first push rod extending proximally from the proximal region of the sheath;
a first engagement feature disposed within the distal region of the sheath; and
a dilator including:
a dilator body including a distal tapered region and a proximal region;
a second push rod extending proximally from the tapered dilator body; and
a second engagement feature disposed within the proximal region of the dilator body, the second engagement feature adapted to releasably engage the first engagement feature.

2. The access system of claim 1, wherein the first engagement feature comprises:
a first marker band secured about the sheath; and
a second marker band spaced from the first marker band and secured about the sheath;
wherein the second marker band is adapted to allow the second engagement feature to move distally past the second marker band.

3. The access system of claim 2, wherein the first marker band has an annular shape and the second marker band has a C-shape.

4. The access system of claim 1, wherein the second engagement feature comprises an annular protrusion on the proximal region of the dilator body.

5. The access system of claim 1, wherein the second engagement feature comprises an O-ring disposed on the proximal region of the dilator body.

6. The access system of claim 1, wherein the second engagement feature comprises one or more bumps protruding from the proximal region of the dilator body.

7. The access system of claim 1, further comprising a push enhancer disposed on the second push rod.

8. The access system of claim 7, wherein the push enhancer is adapted to engage with the proximal region of the sheath.

9. The access system of claim 7, wherein the push enhancer is positioned on the second push rod at a position that prevents the proximal region of the dilator body from moving distally beyond the distal region of the sheath.

10. The access system of claim 7, wherein the push enhancer comprises a proximal collar stop having a distal region adapted to be complementary to a proximal region of the sheath.

11. The access system of claim 7, wherein the second push rod includes one or more bends forming the push enhancer.

12. The access system of claim 1, wherein:
the sheath has a flared distal tip; and
the dilator body is dimensioned to cover the flared distal tip.

13. The access system of claim 12, wherein the dilator body has a hollow portion that allows the dilator body to be pulled within the sheath.

14. The access system of claim 1, further comprising a retainer member that is adapted to be releasably secured to the first push rod and the second push rod.

15. The access system of claim 14, wherein the retainer member comprises a clip.

16. The access system of claim 14, wherein the retainer member comprises a peelable sheath.

17. An access system adapted for accessing a desired location within a patient's vasculature, the access system comprising:
a guide extension catheter including:
a sheath extending from a distal region to a proximal region;
a first push rod extending proximally from the proximal region of the sheath;
a first marker band secured about the sheath; and
a second marker band spaced from the first marker band and secured about the sheath; and
a dilator including:
a dilator body including a distal tapered region and a proximal region;
a second push rod extending proximally from the tapered dilator body; and
a protrusion disposed within the proximal region of the dilator body, the protrusion adapted to be releasably secured between the first marker band and the second marker band.

18. The access system of claim 17, wherein the second marker band has a C-shape that allows the second marker band to flex as the protrusion moves past the second marker band towards the first marker band.

19. An access system adapted for accessing a desired location within a patient's vasculature, the access system comprising:
an outer catheter including:
an outer catheter push rod;
an outer catheter sheath extending distally from the outer catheter push rod;
a first engagement feature disposed within a distal region of the outer catheter sheath; and
an inner catheter including:
an inner catheter push rod;
an inner catheter body extending distally from the inner catheter push rod;
a second engagement feature disposed within a proximal region of the inner catheter body, the second engagement feature adapted to releasably engage the first engagement feature.

20. The access system of claim 19, wherein:
the outer catheter comprises a guide extension catheter; and
the inner catheter comprises a dilator adapted to extend distally from the outer catheter sheath when the inner catheter is advanced through the outer catheter.
